(19) United States
(12) Patent Application Publication
(10) Pub. No.: US 2016/0045506 A1
(43) Pub. Date: Feb. 18, 2016

(54) USE OF INHIBITORS OF BINDING BETWEEN A PAR-1 RECEPTOR AND ITS LIGANDS FOR THE TREATMENT OF GLIOMA

(71) Applicant: UNIVERSITY OF ROCHESTER, Rochester, NY (US)

(72) Inventors: Steven A. GOLDMAN, Webster, NY (US); Romane AUVERGNE, Rochester, NY (US)

(73) Assignee: UNIVERSITY OF ROCHESTER, Rochester, NY (US)

(21) Appl. No.: 14/776,961

(22) PCT Filed: Mar. 14, 2014
(86) PCT No.: PCT/US14/27653

§ 371 Date: Sep. 15, 2015

Related U.S. Application Data
(60) Provisional application No. 61/788,013, filed on Mar. 15, 2013.

(51) Int. Cl. A61K 31/519; A61K 31/4439; A61K 31/443

(52) U.S. Cl. CPC A61K 31/519; A61K 31/443; A61K 31/4439

(57) ABSTRACT

The present invention relates to a method of treating glioma in a subject. The method comprises selecting a subject having a glioma, providing an inhibitor of binding between a PAR-1 receptor and a ligand of the PAR-1 receptor, and administering the inhibitor to the selected subject under conditions effective to treat the glioma and/or prevent spread of tumor cells. Methods for inhibiting proliferation of glioma cells and/or precursors thereof and a method of screening for compounds suitable for treating glioma in subjects are also disclosed.

This application claims the benefit of U.S. Provisional Patent Application Ser. No. 61/788,013, filed Mar. 15, 2013, which is hereby incorporated by reference in its entirety.

### FIELD OF THE INVENTION

The present invention relates to the use of inhibitors of binding between a PAR-1 receptor and its ligands for the treatment of glioma

### BACKGROUND OF THE INVENTION

Malignant gliomas are highly invasive and neurologically destructive tumors, whose most aggressive manifestation is glioblastoma. The term “glioma” encompasses a group of cancers that includes astrocytomas, oligodendrogliomas, oligoastrocytomas, and ependymomas. The most widely used scheme for classification and grading of glioma is that of the World Health Organization, where gliomas are classified according to their hypothesized line of differentiation, that is whether they display features of astrocytic, oligodendrial, or ependymal cells. They are graded on a scale of I to IV according to their degree of malignancies. Glioblastoma (GBM) is classified as grade IV anaplastic astrocytoma.

Despite significant improvements in the early detection of malignant gliomas, the median survival of patients remains less than 12 months from the time of diagnosis (Benedetti et al., “Gene Therapy of Experimental Brain Tumors Using Neural Progenitor Cells,” Nature Med., 6:447-450 (2000); Russell et al., Pathology of Tumors of the Nervous System, (Arnold, Ed.), London (1989)). Malignant gliomas rarely metastasize outside the central nervous system, but they will diffusely invade the host brain. Peritumor brain tissue shows various types of inflammatory responses, including activated macrophages and microglia, hypertrophic reactive astrocytes, vascular invasion and edema formation (Schiffer, D., “Brain Tumors. Biology, Pathology, and Clinical References” Springer, N.Y., Berlin, Heidelberg (1997)). Neurons are preserved in the immediate vicinity of some tumors, but other tumors are surrounded by degenerating neurons, progressing to neuronal loss (Id). Variability in the local presentation of resident neurons has been a frequent but unexplained observation in tumor neuropathology. A similar variable observation has been the incidence of epileptic activity in glioblastoma, which approaches 50% of all cases (Cascino, C., “Epilepsy and Brain Tumors: Implications for Treatment,” Epilepsia, 31: S37-44 (1990); Pallias, J. E., “A Review of 2,413 Tumours Operated Over a 30-year Period,” J. Neuroadiol., 18: 79-106 (1991)).

Glial tumors, the most prevalent and morbid of which is astrocytoma and its aggressive derivative glioblastoma multiforme, are the most common cancers of the adult central nervous system. They are also among the least treatable cancers, with a 5 year survival after initial diagnosis of <10% for tumors initially diagnosed at the grade 3 (anaplastic astrocytoma) or 4 (glioblastoma) stages. Current treatments of glioma and glioblastoma are lacking and achieve only palliation and short-term increments in survival. They include surgical resection—following which ultimate recurrence rates are over 90%—as well as radiation therapy, and chemotherapies that include cis-platin, BCNU and other mitotic inhibitors. The benefits of these current therapies are brief and temporary, and none are curative (e.g., Schiffer. D. Brain Tumors, Biology, Pathology, and Clinical References. Springer-Verlag (New York, Berlin, Heidelberg, 1997).

The present invention is directed to overcoming these and other deficiencies in the art.

### SUMMARY OF THE INVENTION

A first aspect of the present invention relates to a method of treating glioma in a subject. The method comprises selecting a subject having a glioma, providing an inhibitor of binding between a PAR-1 receptor and a ligand of the PAR-1 receptor, and administering the inhibitor to the selected subject under conditions effective to treat the glioma and/or prevent spread of tumor cells.

The present invention is also directed to a method of inhibiting proliferation of glioma cells and/or precursors thereof. The method comprises providing an inhibitor of binding between a PAR-1 receptor and a ligand of the PAR-1 receptor and contacting the inhibitor with glioma cells and/or precursors thereof under conditions effective to inhibit proliferation of the glioma cells and/or precursors thereof.

Another aspect of the present relates to a method of screening for compounds suitable for treating glioma in subjects. The method comprises providing a collection of candidate compounds, providing a PAR-1 receptor, and providing a ligand of the PAR-1 receptor. The collection, the PAR-1 receptor, and the ligand are contacted under conditions effective for the ligand to bind to the PAR-1 receptor in the absence of the collection. Those candidate compounds which inhibit binding between the ligand and the PAR-1 receptor are identified as potential glioma therapeutics.

The protease activated receptor (PAR) family that includes PAR-1 comprises four distinct G-protein coupled receptors, which share a unique mechanism of activation; they are all activated by proteolytic cleavage of their N-terminal ectodomains by serine protease (Coughlin, “Protease-Activated Receptors in Hemostasis, Thrombosis and Vascular Biology,” J Thromb Haemost 3(8): 1800-14 (2005), which is hereby incorporated by reference in its entirety). PAR-1, the prototype of this receptor family, is the principal thrombin-activated receptor in most cell types (Elste and Petersen, “Expression of Proteinase-Activated Receptor 1-4 (Par 1-4) in Human Cancer,” J Mol Histol 41: 89-99 (2010), which is hereby incorporated by reference in its entirety). In addition to its role in blood coagulation and thrombus formation (Coughlin, “Protease-Activated Receptors in Hemostasis, Thrombosis and Vascular Biology,” J Thromb Haemost 3(8): 1800-14 (2005), which is hereby incorporated by reference in its entirety), the thrombin/PAR-1 complex is expressed by a variety of cancers. PAR-1 has been shown to promote the proliferation and survival of tumor cells, as well as tumor angiogenesis and metastasis through endothelial cell activation, in a variety of epithelial malignancies (Bar-Shavit et al., “Parl Plays a Role in Epithelial Malignancies: Transcriptional Regulation and Novel Signaling Pathway,” IUBMB Life 63:397-402 (2011); Coughlin, “Thrombin Signalling and Protease-Activated Receptors,” Nature 407:258-264 (2000); Garcia-Lopez et al., “Thrombin-Activated Receptors: Promising Targets for Cancer Therapy?” Curr Med Chem 17:109-128 (2010), which are hereby incorporated by reference in their entirety). Among neuroectodermal cancers, PAR-1 has been recently proposed as a therapeutic target in melanoma (Villares et al., “The Emerging Role of the Thrombin Receptor (Par-1) in Melanoma Metastasis—a Possible Therapeutic Target,” Oncotarget 2:8-17 (2011); Zigler et al., “Par-1 and Thrombin: The Ties That Bind the Microenvironment to Melanoma Metastasis,” Cancer Res 71:6561-6566 (2011), which are hereby incorporated by reference in their entirety). In glioma, PAR-1 has been shown to be over-expressed by most primary tumors, and its level of expression has been reported to correlate with higher grade, predicting low Karnovsky performance score and poor prognosis (Zhang et al., “Upregulation of Matrix Metalloproteinase-1 and Proteinase-Activated Receptor-1 Promotes the Progression of Human Gliomas,” Pathol Res Pract 207:24-29 (2011), which is hereby incorporated by reference in its entirety).

On that basis, in the present study the necessity of PAR-1 to glioma progression was investigated, and it was found that lentiviral shRNAi knock-down of PAR-1 inhibited the expansion and proliferation of glioma TPCs in vitro, and potently suppressed their tumorigenic potential in vivo after orthotopic transplantation into the brain of immunodeficient mice. It was then found that the thrombin inhibitor dabigatran etexilate (dabigatran), as well as the selective PAR-1 antagonists SCH79797 and SCH530348, each suppressed the growth and proliferation of glioma TPCs in vitro. On that basis, the antineoplastic activity of dabigatran was then assessed in vivo, in mice bearing subcutaneous human glioma TPC xenograft, and it was found that dabigatran significantly reduced tumor volumes compared to vehicle controls. Together, these data reveal the thrombin/PAR-1 axis as a potential new therapeutic target for the treatment of malignant gliomas.

### BRIEF DESCRIPTION OF THE DRAWINGS

FIGS. 1A-1C show PAR-1 is overexpressed in glioma tumor precursor cells (TPCs). FIG. 1A is a table summarizing the expression of PAR-1 mRNA in A2B5⁺glioma TPCs. Microarray analysis of A2B5⁺ sorted cells from primary gliomas, relative to their non-tumor glial precursor cells A2B5⁺ homologues (GPCs), and/or unsorted (UNS) normal cells derived from adult human white matter and cortex revealed PAR-1 mRNA as significantly up-regulated at every stage of glioma progression. The fold-change (FC) and p-value were obtained using a one-way ANOVA followed by post hoc comparisons using Tukey multiple comparison test. To validate the array data, quantitative real-time Taqman RT-PCR was performed using a 96-gene Low Density Array (TLDA), on glioma (n=19) and non-tumor (n=5) A2B5⁺ sorted cells. The expression of each gene was normalized to GAPDH, and p-values were calculated on ΔCₜ values. TLDA gene expression data was analyzed using a moderated t-test statistic with a 5% false discovery rate cut-off. FIG. 1B shows the relative quantification of PAR-1 gene expression using RT-PCR detection in glioma TPC lines (GCLs) established from unsorted cells or A2B5⁺ isolated cells from malignant gliomas. GPC lines were derived from primary GBM and maintained in serum-free media (SFM) supplemented with FGF, EGF (20 ng/ml) and PDGF (10 ng/ml) for less than 10 passages. Comparable quantities of cDNA were ensured by amplification of GAPDH. FIG. 1C shows flow cytometry analysis of PAR-1 protein expression in TPC cell lines cultured in SFM supplemented with FGF, EGF, and PDGF established from A2B5⁺ (375A+, 383A+) and unsorted (233, 238) primary GBM cells, or adherent glioma cells (U87, U251) maintained in 10%/o serum culture conditions. Error bars indicates Means±SEM.

FIGS. 2A-2G show PAR-1 silencing inhibits the growth and proliferation of glioma cells and TPCs. FIG. 2A-2B shows validation of PAR-1 gene (FIG. 2A) and protein (FIG. 2B) induced silencing using quantitative RT-PCR (FIG. 2A) and flow cytometry (FIG. 2B) in glioma cells (U87, U251) and/orA2B5 derived glioma TPC cell lines (GCLs), 6 days after transduction with 3 different PAR-1 knock-down (KD) lentiviruses, compared to glioma cells transduced with a scrambled lentivirus (SCR) and control (CT) untransduced cells. Gene expression levels were normalized to GAPDH. Significance is indicated with *p<0.05; ** p<0.01; ***p<0.001 using one-way ANOVA with repeated measures (P<0.0001) followed by Tukey post-hoc comparisons. Mean±SEM. FIG. 2C shows representative photomicrographs illustrating the number of glioma cells and GCLs 6 days after transduction with either PAR-1-KD or control lentiviruses. Scale bar=100 μm. FIG. 2D-2G shows the effects of PAR-1 silencing on the in vitro expansion (FIG. 2D, FIG. 2F) and proliferation (FIG. 2E, FIG. 2G) of glioma cells (U87, U251) and A2B5⁺ derived glioma TPC lines (GCLs), 6 days after transduction with 3 different PAR-1 knock-down (KD) lentiviruses, compared to glioma cells transduced with a scrambled lentivirus (SCR) and control (CT) untransduced cells. FIG. 2D and FIG. 2F shows lentiviral KD of PAR-1 significantly reduced the number of glioma cells and A2B5+TPCs relative to both SCR shRNAi transduced and non-transduced CT cells. FIG. 2E and FIG. 2G show, using EdU incorporation analysis in association with propidium iodide (PI) staining, that glioma cells and glioma TPCs subjected to PAR-1 KD manifested fewer cells in S phase relative to scrambled transduced and control cells. KD: Knock-down. Means±SEM. P-values calculated using one-way ANOVA with repeated measures (P<0.0001) followed by Tukey post-hoc comparisons with *p<0.05; ** p<0.01; ***p<0.001.

FIGS. 3A-3B show PAR-1 silencing inhibits the in vivo expansion of U87 glioma cells. Effects of PAR-1 silencing are shown on the in vivo expansion of U87 glioma cells, 4 weeks after transduction with PAR-1 knock-down (KD) lentiviruses, compared to glioma cells transduced with a scrambled lentivirus (SCR) and control (CT) untransduced cells. FIG. 3A shows lentiviral KD of PAR-1 significantly reduced tumor formation after intracranial transplantation of U87 cells (80,000 cells/animal) (n=3 per group) into the brain of immunodeficient NOG mice. FIG. 3B shows hematoxylin-eosin stained sections of xenografts following transplantation of U87 cells transduced with PAR-1 KD lentiviruses, SCR lentivirus or CT cells illustrating a potent inhibitory effect of PAR-1 silencing on the tumorigenicity of U87 cells relative to control cells. KD: Knock-down.

FIGS. 4A-4C show PAR-1 silencing inhibits the in vivo expansion of A2B5+TPCs. Effects of PAR-1 silencing are shown on the in vivo expansion of A2B5⁺ TPC isolated from two GBM-derived cell lines cultured in serum-free media supplemented with FGF, EGF (20 ng/ml), and PDGF (10 ng/ml) (28,000 cells/animal, n=3 mice per group for each cell line), 4 weeks after transduction with PAR-1 knock-down (KD) lentiviruses, compared to glioma cells transduced with a scrambled lentivirus (SCR) and control (CT) untransduced cells. FIG. 4A shows hematoxylin-eosin stained sections of xenografts following intracranial implantation of A2B5⁺ TPCs showing a dramatic decrease in the tumorigenic potential of A2B5 TPCs expressing shRNA-PAR-1, relative to both SCR and control cells. FIGS. 4B-4C are graphs representing the stereological analysis of the tumor extension, measured along the antero-posterior axis of xenograft mice brain (FIG. 4B), and the number of xenografted cells stained with the anti-human nuclei antigen (HNA) antibody per mm³ of total brain volume (FIG. 4C), 4 weeks following orthotopic transplantation, demonstrating a prominent inhibitory effect of PAR-1 silencing on the tumorigenicity of glioma A2B5⁺ TPCs relative to SCR and CT cells. KD: Knock-down. Means±SEM. P-values calculated using one-way ANOVA (P<0.0001) followed by Tukey post-hoc comparisons with *p<0.05; ** p<0.01; ***p<0.001.

FIGS. 5A-5D show pharmacological inhibition of PAR-1 inhibits glioma cells and TPCs expansion in vitro. Effects of the specific PAR-1 inhibitor SCH79797 are shown on the in vitro expansion of A2B5⁺ glioma TPCs (GCL) isolated from two GBM-derived cell lines cultured in serum-free media supplemented with FGF, EGF (20 ng/ml), and PDGF (10 ng/ml) (FIG. 5A), adherent glioma cells cultured with 10% serum (U87, U251) (FIG. 5B), and normal astrocytes derived from the fetal (FIG. 5C) and adult human brain (FIG. 5D). SCH79797 significantly decreases the number of GCL and glioma cells in a dose dependent manner, 4 days after administration of the drug, while it did not affect the growth of normal fetal and adult astrocytes. Mean±SEM. P-values calculated using one-way ANOVA with repeated measures followed by Tukey post-hoc comparisons with *p<0.05; ** p<0.01; ***p<0.001.

FIGS. 6A-6C show pharmacological inhibition of PAR-1 inhibits glioma cells and TPCs expansion in vitro. Effects of the oral specific PAR-1 inhibitor SCH530348 are shown on the in vitro expansion of A2B5⁺ glioma TPCs (GCL) isolated from two GBM-derived cell lines cultured in serum-free media supplemented with FGF, EGF (20 ng/ml), and PDGF (1 Ong/ml) (FIG. 6A), adherent glioma cells cultured with 10% serum (U87, U251) (FIG. 6B), and normal astrocytes derived from the fetal (FIG. 6C) and adult human brain (FIG. 6D). SCH530348 significantly decreases the number of GCL and glioma cells in a dose dependent manner, 4 days after administration of the drug, while it did not affect the growth of normal adult astrocytes, and slightly decreases the growth of normal fetal astrocytes for concentrations >0.5 uM. Mean±SEM. P-values calculated using one-way ANOVA with repeated measures followed by Tukey post-hoc comparisons with *p<0.05; ** p<0.01; ***p<0.001.

FIGS. 7A-7D show pharmacological inhibition of thrombin inhibits glioma cells and TPCs expansion in vitro. Effects of the thrombin inhibitor dabigatran etexilate on the in vitro expansion of A2B5⁺ glioma TPCs (GCL) isolated from two GBM-derived cell lines cultured in serum-free media supplemented with FGF, EGF (20 ng/ml), and PDGF (1 Ong/ml) (FIG. 7A), adherent glioma cells cultured with 10% serum (U87, U251) (FIG. 7B), and normal astrocytes derived from the fetal (FIG. 7C) and adult human brain (FIG. 7D). Dabigatran etexilate significantly decreases the number of GCL and glioma cells in a dose dependent manner, 4 days after administration of the drug, while it did not affect the growth of normal adult astrocytes, and slightly decreases the growth of normal fetal astrocytes for concentrations >0.5 uM. Mean±SEM. P-values calculated using one-way ANOVA with repeated measures followed by Tukey post-hoc comparisons with *p<0.05; ** p<0.01; ***p<0.001.

FIG. 8 shows pharmacological inhibition of thrombin inhibits the tumor initiation and maintenance potential of TPCs in vivo. Effects of the oral thrombin inhibitor dabigatran etexilate (Pradaxa) are shown on the in vivo expansion of A2B5 glioma TPCs (GCL) isolated from GBM-derived cell line cultured in serum-free media supplemented with FGF, EGF (20 ng/ml), and PDGF (10 ng/ml), after subcutaneous injection into the flank of immunodeficient NOG mice (2.5E⁺⁰⁶ cells/animal, n=6 mice), and treated twice daily with Pradaxa (37.5 mg/kg, twice daily, oral gavage) or vehicle (n=3 animals per group), starting 11 days post-subcutaneous injection. Pradaxa significantly reduce the tumor volumes compared to vehicle controls. Mean±SEM. P-values calculated using one-way ANOVA with repeated measures followed by Tukey post-hoc comparisons with *p<0.05; ** p<0.01; ***p<0.001.

### DETAILED DESCRIPTION OF THE INVENTION

A first aspect of the present invention relates to a method of treating glioma in a subject. The method comprises selecting a subject having a glioma, providing an inhibitor of binding between a PAR-1 receptor and a ligand of the PAR-1 receptor and administering the inhibitor to the selected subject under conditions effective to treat the glioma and/or prevent spread of tumor cells.

As used herein, the term “glioma” refers to a tumor that arises from glial cells or their precursors of the brain or spinal cord. Gliomas are histologically defined based on whether they exhibit primarily astrocytic or oligodendroglial morphology, and are graded by cellularity, nuclear atypia, necrosis, mitotic figures, and microvascular proliferation—all features associated with biologically aggressive behavior. Astrocytomas are of two main types—high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord.

As used herein, the inhibition of binding between a PAR-1 receptor and a ligand of the PAR-1 receptor includes inhibition of any one of: direct physical binding between a PAR-1 receptor and a ligand of the PAR-1 receptor; inhibition of proteolysis of the PAR-1 receptor by a ligand; or inhibition of PAR-1 receptor targeted protease.

The inhibitor of binding between a PAR-1 receptor and a ligand of the PAR-1 receptor may include any of the following: peptide inhibitors, peptide mimetic inhibitors, small molecule compound inhibitors, and antibodies or binding portions thereof.

Exemplary peptide inhibitors include, but are not limited to: BMS-197525 (Bernatowicz et al., “Development of Potent Thrombin Receptor Antagonist Peptides,” J Med Chem 39:4879-4887 (1996), which is hereby incorporated by reference in its entirety); photoactivatable peptides based on BMS-197525 (Elliott et al., “Photoactivatable Peptides Based on BMS-197525: A Potent Antagonist of the Human Thrombin Receptor (PAR-1),” Bioorganic & Medicinal Chemistry Letters 9:279-284 (1999), which is hereby incorporated by reference in its entirety); RPPGF (Hasan et al., “Mechanisms of Arg-Pro-Pro-Gly-Phe Inhibition of Thrombin,” Am J Physiol Heart Circ Physiol 285:H183-H193 (2003), which is hereby incorporated by reference in its entirety); FLLRN (Vassallo et al., “Structure-Function Relationships in the Activation of Platelet Thrombin Receptors by Receptor-derived Peptides,” J Biol Chem 267(9):6081-6085 (1992), which is hereby incorporated by reference in its entirety); BMS-200261 (Bernatowicz et al., “Development of Potent Thrombin Receptor Antagonist Peptides,” J Med Chem 39:4879-4887 (1996), which is hereby incorporated by reference in its entirety); BMS-200661 (Bernatowicz et al., “Development of Potent Thrombin Receptor Antagonist Peptides,” J Med Chem 39:4879-4887 (1996), which is hereby incorporated by reference in its entirety); MSRPACN (Doorbar and Winter, “Isolation of a Peptide Antagonist to the Thrombin Receptor Using Phage Display,” J Mol Biol 244(4):361-369 (1994), which is hereby incorporated by reference in its entirety); and recombinant hirudin and hirulogs (Coppens et al., “Translational Success Stories: Development of Direct Thrombin Inhibitors,” Circ Res 111(7):920-929 (2012), which is hereby incorporated by reference in its entirety).

Exemplary peptide mimetic inhibitors include, but are not limited to: RWJ-56110 (Andrade-Gordon et al., “Design, Synthesis, and Biological Characterization of a Peptide-Mimetic Antagonist for a Tethered Ligand Receptor,” Proc Natl Acad Sci 96(22): 12257-12262 (1999), which is hereby incorporated by reference in its entirety); RWJ-58259 (Zhang et al., “Discovery and Optimization of a Novel Series of Thrombin Receptor (PAR1) Antagonists: Potent, Selective Peptide Mimetics Based on Indole and Indazole Templates,” J Med Chem 44:1021-1024 (2001), which is hereby incorporated by reference in its entirety); RWJ-53052 (Zhang et al., “Discovery and Optimization of a Novel Series of Thrombin Receptor (PAR1) Antagonists: Potent, Selective Peptide Mimetics Based on Indole and Indazole Templates,” J Med Chem 44:1021-1024 (2001), which is hereby incorporated by reference in its entirety); and P1 pal-12 (Kubo et al., “Distinct Activity of Peptide Mimetic Intracellular Ligands (Pepducins) for Proteinase-Activated Receptor-1 in Multiple Cells/Tissues,” Ann NY Acad Sci 1091:445-459 (2006), which is hereby incorporated by reference in its entirety).

Exemplary small molecule compound inhibitors include, but are not limited to: SCH 530348 (Bonaca and Morrow, “SCH 530348: A Novel Oral Thrombin Receptor Antagonist,” Future Cardiol 5(5):435-442 (2009), which is hereby incorporated by reference in its entirety); E5555 (Rollini et al., “Atopaxar: A Review of its Mechanism of Action and Role in Patients With Coronary Artery Disease,” Future Cardiol 8(4):503-511 (2012), which is hereby incorporated by reference in its entirety); F16618 (Perez et al., “Discovery of Novel Protease Activated Receptors 1 Antagonists with Potent Antithrombotic Activity In Vivo,” J Med Chem 52:5826-5836 (2009), which is hereby incorporated by reference in its entirety); F16357 (Perez et al., “Discovery of Novel Protease Activated Receptors 1 Antagonists with Potent Antithrombotic Activity In Vivo,” J Med Chem 52:5826-5836 (2009), which is hereby incorporated by reference in its entirety); 1,3-Diaminobenzenes (Dockendorff et al., “Discovery of 1,3-Diaminobenzenes as Selective Inhibitors of Platelet Activation at the PAR1 Receptor,” ACS Medicinal Chemistry Letters 3:232-237 (2012), which is hereby incorporated by reference in its entirety); heterotricyclic himbacine analogs (Chelliah et al., “Heterotricyclic Himbacine Analogs as Potent, Orally Active Thrombin Receptor (Protease Activated Receptor-1) Antagonists,” J Med Chem 50:5147-5160 (2007), which is hereby incorporated by reference in its entirety); nor-seco himbacine analogs (Chelliah et al., “Discovery of Nor-Seco Himbacine Analogs as Thrombin Receptor Antagonists,” Bioorganic & Medicinal Chemistry Letters 22:2544-2549 (2012), which is hereby incorporated by reference in its entirety); ER-121958-06 (Bocquet et al., “Effects of a New PAR1 Antagonist, F 16618, On Smooth Muscle Cell Contraction,” Eur J Pharmacol 611:60-63 (2009), which is hereby incorporated by reference in its entirety); KC-AOY (Lee, “Discovery of an Orally Available PAR-1 Antagonist as a Novel Antiplatelet Agent,” Arch Pharm Res 34(4):515-517 (2011), which is hereby incorporated by reference in its entirety); FR171113 (Kato et al., “Inhibition of Arterial Thrombosis By a Protease-Activated Receptor 1 Antagonist, FR171113, in the Guinea Pig,” Eur J Pharmacol 473(2-3): 163-169 (2003), which is hereby incorporated by reference in its entirety); SCH 203099 and SCH 79797 (Ahn et al., “Inhibition of Cellular Action of Thrombin by N3-Cyclopropyl-7-[[4-(1-Methylethyl)Phenyl]Methyl]-7H-Pyrrolo[3,2-f]Quinazoline-1,3-Diamine (SCH 79797), A Nonpeptide Thrombin Receptor Antagonist,” Biochem Pharmacol 60(10): 1425-1434 (2000), which is hereby incorporated by reference in its entirety); SCH 602539 (Chintala et al., “SCH 602539, A Protease-Activated Receptor-1 Antagonist, Inhibits Thrombosis Alone and In Combination With Cangrelor in a Folts Model of Arterial Thrombosis in Cynomolgus Monkeys,” Arterioscler Thromb Vasc Biol 30(11):2143-2149 (2010), which is hereby incorporated by reference in its entirety); benzimidazole derivatives (Chackalamannil et al., “Potent, Low Molecular Weight Thrombin Receptor Antagonists,” Bioorganic & Medicinal Chemistry Letters 11:2851-2853 (2001), which is hereby incorporated by reference in its entirety); pyrroloquinazolines (Ahn et al., “Structure-Activity Relationships of Pyrroloquinazolines as Thrombin Receptor Antagonists,” Bioorganic & Medicinal Chemistry Letters 9:2073-2078 (1999), which is hereby incorporated by reference in its entirety); a “Merck isoxazole” (Nantermet et al., “Discovery of a Nonpeptidic Small Molecule Antagonist of the Human Platelet Thrombin Receptor (PAR-1),” Bioorganic & Medicinal Chemistry Letters 12:319-323 (2002), which is hereby incorporated by reference in its entirety); argatroban (Coppens et al., “Translational Success Stories: Development of Direct Thrombin Inhibitors,” Circ Res 111(7):920-929 (2012), which is hereby incorporated by reference in its entirety); ximelagatran (Coppens et al., “Translational Success Stories: Development of Direct Thrombin Inhibitors,” Circ Res 111(7):920-929 (2012), which is hereby incorporated by reference in its entirety); and dabigatran etexilate (Coppens et al., “Translational Success Stories: Development of Direct Thrombin Inhibitors,” Circ Res 111(7):920-929 (2012), which is hereby incorporated by reference in its entirety).

Inhibitory PAR-1 antibodies have also been described in the art and are commercially available. These antibodies include WEDE15 and ATAP2, which are available from Beckman Coulter and Santa Cruz Biotechnology, respectively.

In a preferred embodiment, the glioma is located within the brain of the subject. These gliomas may exist, without limitation, within the frontal lobe, temporal lobe, parietal lobe, occipital lobe, brain stem, and cerebellum.

In another preferred embodiment, the glioma is located in the spinal cord of the subject. These tumors may arise within the spinal cord itself, spinal nerve roots, or dura.

Whether the glioma is located in the brain or spinal cord, the glioma is classified based on a histological grade. Histological grading is used to predict the biological behavior of the tumor and can affect overall survival rate. Grade I gliomas encompasses tumors with low proliferative potential and the possibility of a cure after surgical resection. Grade II gliomas are usually infiltrative, have low proliferative activity, but tend to recur. Grade III gliomas contain evidence of malignancy, including nuclear abnormalities and increased proliferation. Grade IV gliomas are cytologically malignant, highly proliferative tumors associated with disease progression despite surgical resection and a fatal outcome (Louis et al., “The 2007 WHO Classification of Tumours of the Central Nervous System,” Acta Neuropathol 114: 97-109 (2007), which is hereby incorporated by reference in its entirety).

In one embodiment, the glioma is a Grade I glioma. Examples of Grade I gliomas, based on the 2007 WHO classification of tumors of the central nervous system, include, without limitation, subependymal giant cell astrocytomas, pilocytic astrocytomas, subependymomas, and myxopapillary ependymomas (Louis et al., “The 2007 WHO Classification of Tumours of the Central Nervous System,” Acta Neuropathol 114: 97-109 (2007), which is hereby incorporated by reference in its entirety).

In another embodiment, the glioma is a Grade II glioma. Examples of Grade II gliomas include, without limitation, pilomyxoid astrocytomas, diffuse astrocytomas, pleomorphic xanthoastrocytomas, oligodendrogliomas, oligoastrocytomas, and ependymomas (Louis et al., “The 2007 WHO Classification of Tumours of the Central Nervous System,” Acta Neuropathol 114: 97-109 (2007), which is hereby incorporated by reference in its entirety).

In a yet another embodiment, the glioma is a Grade III glioma. Examples of Grade III gliomas include, without limitation, anaplastic astrocytomas, anaplastic oligodendrogliomas, and anaplastic ependymomas (Louis et al., “The 2007 WHO Classification of Tumours of the Central Nervous System,” Acta Neuropalhol 114: 97-109 (2007), which is hereby incorporated by reference in its entirety).

In another embodiment, the glioma is a Grade IV glioma. Grade IV gliomas include, without limitation, glioblastoma, giant cell glioblastoma, and gliosarcoma (Louis et al., “The 2007 WHO Classification of Tumours of the Central Nervous System,” Acta Neuropathol 114: 97-109 (2007), which is hereby incorporated by reference in its entirety).

The subject having the glioma can be any mammal (e.g. mouse, rat, rabbit, hamster, guinea pig, cat, dog, pig, goat, cow, horse, primate, or human). Preferably, the subject is a human.

The inhibitor of binding between a PAR-1 receptor and a ligand of the PAR-1 receptor used according to the methods of the present invention can be administered alone or as a pharmaceutical composition. Preferably, the inhibitor is present in a pharmaceutical composition comprising the inhibitor and a pharmaceutically acceptable carrier.

The pharmaceutical composition of the present invention can further contain other pharmaceutically acceptable components (see REMINGTON'S PHARMACEUTICAL SCIENCE (19th ed., 1995), which is hereby incorporated by reference in its entirety). The incorporation of such pharmaceutically acceptable components depends on the intended mode of administration and therapeutic application of the pharmaceutical composition. As described above, preferably, the pharmaceutical composition will include a pharmaceutically-acceptable, non-toxic carrier or diluent, which are defined as vehicles commonly used to formulate pharmaceutical compositions for animal or human administration. The diluent is selected so as not to affect the biological activity of the composition. Exemplary carriers or diluents include distilled water, physiological phosphate-buffered saline, Ringer's solutions, dextrose solution, and Hank's solution.

Pharmaceutical compositions can also include large, slowly metabolized macromolecules such as proteins, polysaccharides such as chitosan, polylactic acids, polyglycolic acids and copolymers (such as latex functionalized sepharose, agarose, cellulose), polymeric amino acids, amino acid copolymers, and lipid aggregates (such as oil droplets or liposomes).

The pharmaceutical composition may also further include a delivery vehicle. Suitable delivery vehicles include, but are not limited to biodegradable microspheres, microparticles, nanoparticles, liposomes, collagen minipellets, and cochleates.

In a preferred embodiment, compositions of the present invention are administered orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

If the inhibitor is formulated for parenteral administration, solutions or suspensions of the inhibitor can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, propylene glycol or polyethylene glycol, are preferred liquid carriers, particularly for injectable solutions. Under ordinary conditions of storage and use, these preparations contain a preservative to prevent growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the pharmaceutical agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the compositions of the present invention may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

It is also contemplated that administration of the inhibitor of binding between a PAR-1 receptor and a ligand of the PAR-1 receptor can be carried out in combination with other suitable therapeutic treatments which are useful for treating glial tumors. These treatments include, but are not limited to, surgical resection, external beam radiation, stereotactic radiosurgery, fractionated stereotactic radiotherapy, and chemotherapy.

Effective amounts of the inhibitor of binding between a PAR-1 receptor and a ligand of the PAR-1 receptor will depend upon the mode of administration, frequency of administration, nature of the treatment, age and condition of the subject to be treated, and the type of pharmaceutical composition used to deliver the compound into a living system. For inhibitors that are involved in clinical trials or inhibitors that are FDA approved for other indications, the safe and effective dosages identified in such trials can be considered when selecting dosages for treatments according to the present invention.

Administering the inhibitor under conditions effective to treat the glioma may result in observable and/or measurable reduction in or absence of one or more of the following: reduction in the number/absence of glioma cells; reduction in the tumor size; inhibition (i.e., slow to some extent and preferably stop) of glioma cell infiltration into peripheral organs including the spread of glioma into soft tissue and bone; inhibition (i.e., slow to some extent and preferably stop) of glioma metastasis; inhibition, to some extent, of tumor growth; and/or relief to some extent, one or more of the symptoms associated with the specific cancer; reduced morbidity and mortality, and improvement in quality of life issues.

Another aspect of the present invention is directed to a method of inhibiting proliferation of glioma cells and/or precursors thereof. This method comprises providing an inhibitor of binding between a PAR-1 receptor and a ligand of the PAR-1 receptor and contacting the inhibitor with glioma cells and/or precursors thereof under conditions effective to inhibit proliferation of the glioma cells and/or precursors thereof.

In accordance with this aspect of the present invention, precursors of glioma cells include glioma stem cells and glioma progenitor cells.

In one embodiment, said method inhibits proliferation of glioma stem cells. These cells include cells presenting at least one, and preferably all, of: (i) the ability to self-renew in defined stem cell culture conditions, in particular neural stem cell culture conditions; (ii) the expression of normal neural stem cell markers, (iii) the ability to generate a differentiated progeny; and (iv) the capacity to generate brain tumors in animals.

In another embodiment, said method inhibits proliferation of glioma progenitor cells. Glioma progenitor cells are more differentiated than a stem cell and often express specific cell markers. It is believed that such progenitor cells may give rise to the glioma stem cells discussed above (Stiles and Rowitch, “Glioma Stem Cells: a Midterm Exam,” Neuron 58:832-846 (2008), which is hereby incorporated by reference in its entirety).

The inhibitor of binding between a PAR-1 receptor and a ligand of the PAR-1 receptor may be, without limitation, any of the inhibitors described above.

As used herein, “contacting the inhibitor with glioma cells and/or precursors thereof” refers to bringing said inhibitor and glioma cells and/or precursors thereof together, whether in an in vitro system or an in vivo system.

Inhibition of proliferation of the glioma cells and/or precursors thereof can be determined by comparing the difference in proliferation between contacted cells and corresponding controls after measuring the volume of the cells, taking advantage of standard techniques known in the art. This determination may be performed at various points in time, for example, 15 minutes, 30 minutes, 60 minutes, 2 hours, 5 hours, 18 hours, 24 hours, 2 days, 3 days, 4 days, 5 days, six days and/or seven days after contacting said cells with the inhibitor of binding between a PAR-1 receptor and a ligand of the PAR-1 receptor. It is envisaged here that this determination may be performed repeatedly, for example, at 15 minutes, 30 minutes, and 60 minutes after said contacting. It is of note that the cells may be contacted not only once with the inhibitor or exposed to the inhibitor but several times (e.g. 2 times, 3 times, 5 times, 10 times, or 20 times) under various conditions (e.g. same concentration of inhibitor, different concentration of inhibitor, inhibitor comprised in a composition with different stabilizers, diluents, and/or carriers and the like). Accordingly, repeated evaluation/determination may be performed after the final contacting with the inhibitor or in between the above-mentioned various contacting steps. A person skilled in the art will be aware of assays or medical imaging techniques that evaluate the induction of apoptotic cell death, senescence or any other cell biology phenotype that is associated with decreased viability or proliferation of tumor cells.

A final aspect of the present invention is directed to a method of screening for compounds suitable for treating glioblastoma in subjects. The method comprises providing a collection of candidate compounds, providing a PAR-1 receptor, and providing a ligand of the PAR-1 receptor. The collection, the PAR-1 receptor, and the ligand are contacted under conditions effective for the ligand to bind to the PAR-1 receptor in the absence of the collection. Those candidate compounds which inhibit binding between the ligand and the PAR-1 receptor are identified as potential glioma therapeutics.

The candidate compounds utilized in the assays described herein may be essentially any compound suspected of being capable of affecting biological functions or interactions. Exemplary compounds include, without limitation, peptides, peptide mimetics, small molecules, and antibodies. The compound may be part of a library of compounds. Alternatively, the compound may be designed specifically to interact or interfere with the biological activity of PAR-1.

The PAR-1 receptor may be provided in the form of cells that are known to express PAR-1, such as platelets, or, for the purposes of the present invention, glioma cell lines such as U87, U251, or A2B5+ derived glioma stem cell line. PAR-1 may also be purified directly from a biological source, such as human platelets. Methods for purifying proteins from cells are well known in the art.

Alternatively, DNA encoding PAR-1, or fragments thereof, may be operably linked to genetic constructs, e.g., vectors and plasmids for expression in a prokaryotic host. In some cases a nucleic acid is operably linked to a transcription and/or translation sequence in an expression vector to enable expression of a PAR-1 polypeptide. By “operably linked,” it is meant that a selected nucleic add, e.g., a coding sequence, is positioned such that it has an effect on, e.g., is located adjacent to, one or more sequence elements, e.g., a promoter and/or ribosome binding site (Shine-Dalgarno sequence), which directs transcription and/or translation of the sequence. Some sequence elements can be controlled such that transcription and/or translation of the selected nucleic acid can be selectively induced. Exemplary sequence elements include inducible promoters such as tac, T7, P(BAD), (araBAD), and beta-D-glucuronidase (uidA) promoter-based vectors. Control of inducible promoters in E. coli can be enhanced by operably linking the promoter to a repressor element such as the lac operon repressor. In the specific case ofa repressor element, “operably linked” means that a selected promoter sequence is positioned near enough to the repressor element that the repressor inhibits transcription from the promoter (under repressive conditions). Typically, expression plasmids and vectors include a selectable marker (e.g., antibiotic resistance gene such as Tet(R) or Amp(R)). Selectable markers are useful for selecting host cell transformants that contain a vector or plasmid. Selectable markers can also be used to maintain (e.g., at a high copy number) a vector or plasmid in a host cell. Alternatively, the recombinant expression vector can be transcribed and translated in vitro, for example using T7 promoter regulatory sequences and T7 polymerase.

The polypeptide sequence of interest may be expressed as part of a fusion protein using recombinant DNA technology. Fusion vectors add a number of amino acids to a protein encoded therein, usually to the amino terminus of the recombinant protein. Such fusion vectors typically serve three purposes: 1) to increase expression of recombinant protein: 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa or enterokinase. Typical fusion expression vectors include pGEX (Pharmacia Biotech Inc), pMAL (New England Biolabs, Beverly, Mass.), and pRITδ (Pharmacia. Piscataway, N.J.) which fuse glutathione S-transferase (GST), maltose E binding protein, or protein A, respectively, to the target recombinant protein.

The PAR-1 polypeptide may also be engineered to have an affinity tag fused to its N or C terminal end. For example, the target protein may be fused to a short tag peptide, such as the Hexa-His peptide or the HA Epitope Tag (Influenza Hemagglutinin) synthetic peptide YPYDVPDYA (SEQ ID NO: 1). These tag proteins or peptides also facilitate subsequent protein purification by affinity chromatography.

Other commonly used bacterial host plasmids include pUC series of plasmids and commercially available vectors, e.g., pAT153, pBR, PBLUESCRIPT, pθS, pGEM, pCAT, pEX, pT7, pMSG, pXT, pEMBL. Another exemplary plasmid is pREV2.1. Plasmids that include a nucleic add described herein can be transfected or transformed into bacterial host cells for expression of PAR-1 polypeptides. Techniques for transformation are known in the art, including calcium chloride or electroporation. The recombinant DNA sequence can also be cloned into a bacteriophage vector. In certain embodiments, transformed host cells include non-pathogenic prokaryotes capable of highly expressing recombinant proteins. Exemplary prokaryotic host cells include laboratory and/or industrial strains of E. coli cells, such as BL21 or K12-derived strains (e.g., C600, OHIalpha, DH5alpha, HBIOI, INVI, JM109, TBI, TGI, and XLI-Blue). Such strains are available from the ATCC or from commercial vendors such as BO Biosciences Clontech (Palo Alto, Calif.) and Stratagene (La Jolla, Calif.). For detailed descriptions of nucleic acid manipulation techniques, see SAMBROOK AND RUSSELL, MOLECULAR CLONING: A LABORATORY MANUAL (Cold Spring Harbor Press, NY, 2001), which is hereby incorporated by reference in its entirety.

The PAR-1 protein or fragments thereof may be expressed in eukaryotic cells. Using standard recombinant DNA techniques, a PAR-1 nucleic acid is cloned into a vector in a form suitable for expression of the nucleic acid in a host cell. Preferably the recombinant expression vector includes one or more regulatory sequences operatively linked to the nucleic acid sequence that facilitates expression within the eukaryotic cell. The term “regulatory sequence” includes promoters, enhancers, and other expression control elements (e.g., polyadenylation signals). Regulatory sequences include those, which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., PAR-1 proteins, mutant forms of PAR-1 proteins, fusion proteins, and the like). For example, PAR-1 polypeptides can be expressed in insect cells (e.g., using baculovirus expression vectors), yeast cells, or mammalian cells.

In mammalian cells, the expression vector's control functions can be provided by viral regulatory elements, For example, commonly used promoters are derived from polyoma, Adenovirus 2, cytomegalovirus, and Simian Virus 40. In some embodiments, the promoter may be an inducible promoter, e.g., a promoter regulated by a steroid hormone, by a polypeptide hormone (e.g., by means of a signal transduction pathway), or by a heterologous polypeptide (e.g., the tetracycline-inducible systems, Tet-On and Tet-Off). The PAR-1 polypeptide sequence may also comprise a signal peptide sequence which promotes the secretion of the PAR-1 polypeptide.

Mammalian cell lines suitable for protein expression include, but are not limited to, Chinese hamster ovary cells (CHO) or COS cells (African green monkey kidney cells CV-1 origin SV40 cells). Other suitable host cells are known to those skilled in the art.

Vector DNA can be introduced into host cells via conventional transfection techniques. As used herein, the terms “transformation” and “transfection” are intended to refer to a variety of art-recognized techniques for introducing foreign nucleic acid (e.g., DNA) into a host cell, including calcium phosphate co-precipitation, DEAE-dextran-mediated transfection, lipofection, or electroporation.

The ligand of PAR-1 is generated upon thrombin cleavage of the N-terminal domain of PAR-1, removing approximately 15 amino acids. The remaining N-terminal domain, referred to as the tethered ligand, is responsible for the initiation of PAR-1 mediated signaling. Therefore, a peptide corresponding to the naturally occurring tethered ligand peptide can be generated using peptide synthesis methods well known in the art. In addition, generation of the tethered ligand can be induced in PAR-1 expressing cells via the addition of thrombin.

Contacting the collection, the PAR-1 receptor, and the ligand can thus be performed in cell-based and cell-free systems.

In a cell-based system, for example, the cleavage of PAR-1 by thrombin results in a rapid and transient calcium flux in cells that can be measured using commercially available reagents (e.g., commercially available from Molecular Devices). Specifically, candidate compounds can be evaluated using cells that express PAR-1, e.g., HT-29, HCT-116, or DU145 cells, for their ability to inhibit calcium flux after thrombin treatment. Calcium flux can be measured using any method known in the art. In one example, cells in FlipR dye (Molecular Devices, Sunnyvale, Calif.) are pre-incubated with candidate compounds and Ca²⁺ flux is induced with thrombin at various concentrations. Control cells are not pre-incubated with compound. Thrombin-induced calcium flux will be detectably inhibited or decreased in cells that are pre-incubated with a candidate compound of the invention in comparison to thrombin-induced calcium flux in control cells if the compound acts as an inhibitor of binding between the ligand and the PAR-1 receptor. For example, thrombin-induced calcium flux will be decreased at least about 10%, for example, at least 30%, 50%, or 80%, or completely inhibited in cells exposed to the candidate compound in comparison to control cells.

In a cell-free assay, a reaction mixture of a PAR-1 receptor, the PAR-1 ligand, and the candidate compound is prepared under conditions and for a time sufficient to allow the PAR-1 receptor and the PAR-1 ligand to interact and bind, thus forming a complex that can be removed and/or detected. The interaction between two molecules can also be detected, e.g., using fluorescence energy transfer (FRET) (see, for example, U.S. Pat. No. 5,631,169 to Lakowicz et al.; U.S. Pat. No. 4,868,103 to Stavrianopoulos et al., which are hereby incorporated by reference in their entirety). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternatively, the ‘donor’ protein molecule may simply utilize the natural fluorescent energy oftryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed, in a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter). Consequently, it can be determined if the presence of the candidate compound was able to inhibit binding between the ligand and the PAR-1 receptor.

Candidate compounds that are determined to inhibit binding between the PAR-1 ligand and the PAR-1 receptor are identified as potential glioma therapeutics.

### EXAMPLES

The following examples are provided to illustrate embodiments of the present invention but they are by no means intended to limit its scope.

### Materials and Methods for Examples 1-6

Cell and Tissue Processing.

Human tissue samples were obtained from patients who consented to tissue use under protocols approved by the University of Rochester-Strong Memorial Hospital Research Subjects Review Board. Tissue from adult cerebral cortex, subcortical white matter and hippocampus resected for intractable epilepsy were used as controls. Tumors were graded by the attending neuropathologist in accordance with World Health Organization (WHO) established guidelines. Tumor samples were divided into 3 segments: one was prepared for culture, a second was frozen in liquid nitrogen for molecular analysis, and a third was fixed in 4% PFA for immunohistological validation of phenotype.

Sample Preparation.

Samples were minced and digested by papain and DNase I in PIPES buffer, for 1 to 1.5 hours at 37° C. The samples were spun at 200 g and their pellets recovered in 2 ml of Dulbecco's minimum essential medium (DMEM)/F-12/N1. They were then dissociated by sequential trituration and passed through a 40 μm mesh into DMEM/F-12/N1 supplemented with 10% plasma-derived (PD) FBS (Cocalico Biologicals, Reamstown, Pa.) to stop the dissociation. The cell suspension was then diluted in 20 ml of DMEM/F12, mixed with 10 ml of Percoll in PBS, and fractionated by centrifugation at 15,000 g for 20 minutes. Cell fractions were harvested and washed in DMEM/F12. Cells were resuspended in DMEM/F-12/N1 media supplemented with bFGF (20 ng/ml. Sigma), EGF (20 ng/ml), PDGF-AA (20 ng/ml; Sigma), and plated in cell suspension culture dish for overnight recovery. Non-neoplastic GPCs were isolated from matched grey and white matter dissociates of temporal lobes taken from 4 patients (temporal lobe epilepsy, ages 30-46 years). Separation of A2B5 and A2B5⁻ cells was performed by MACS 24-48 hours after dissociation.

Cell Sorting.

Glioma and non-neoplastic A2B5⁺ GPCs were isolated using magnetic-beads (MACS) cell sorting followed by confirmation of the purity of the separated cell populations, as previously described (Nunes et al., “Identification and Isolation of Multipotential Neural Progenitor Cells from the Subcortical White Matter of the Adult Human Brain,” Nat Med 9:439-447 (2003), which is hereby incorporated by reference in its entirety). Briefly, cells were suspended in DMEM/F12/N1 and incubated in A2B5 antibody supernatant (clone 105; American Type Culture Collection, Manassas, Va.) for 30 to 45 minutes at 4° C. on a shaker. The cells were washed three times with phosphate-buffered saline containing 0.5% bovine serum albumin and 2 mM EDTA, then incubated with 1:4 diluted microbead-tagged rat anti-mouse IgM antibody (Miltenyi Biotech, Auburn, Calif.) (MACS) for 30 minutes at 4° C. The A2B5 stained cells were washed, resuspended, and separated by MACS using either MS/RS or LSNS positive selection columns (Miltenyi). Cell viability was determined both before and after sorting, using calcein (Molecular Probes, Eugene, Oreg.).

Establishment of Glioma TPC Lines.

After dissociation, unsorted and A2B5 selected primary cells derived from tumor samples were seeded in a 6 well cell suspension plate at 100,000-200,000 cells/ml in DMEM/F12/N1 media containing FGF, EGF and PDGF (20 ng/ml) as previously described (Nunes et al., “Identification and Isolation of Multipotential Neural Progenitor Cells from the Subcortical White Matter of the Adult Human Brain,” Nat Med 9:439-447 (2003), which is hereby incorporated by reference in their entirety). Growth factors and media were renewed 3 times per week. These cells were clonogenic in vitro, expressed stem/progenitor cell markers, and demonstrated neuronal and/or glial antigenic expression upon phenotypic differentiation. The cells were tumorigenic in vivo after transplantation into the brains of immunodeficient mice, in which they generated tumors that recapitulated the histological and phenotypic features of the parental GBM.

Flow Cytometry.

For flow cytometry, 100,000 cells were resuspended in 100 μl of flow cytometry (FC) buffer (PBS with 2 mM EDTA and 0.5% BSA) and incubated for 15 minutes on ice with the anti-PAR-1 antibody (mouse IgG 1; Clone WEDE15; Fisher Scientific; 1/100). Cells were washed in FC buffer, and incubated with secondary fluorescent-conjugated antibodies (goat anti-mouse IgGI-Alexa 647 conjugated; 1/700) staining for 15 min on ice. Cells were washed once and resuspended in FC buffer supplemented with 4′,6-diamidino-2-phenylindole (DAPI, Invitrogen) to a final concentration of 80 ng/ml before analysis. Control conditions included unlabeled cells and cells labeled with appropriate isotypes control or secondary antibodies alone. Cells were analyzed on a FACS ARIA flow cytometer (BD Biosciences) using the FACS DIVA software and/or FlowJo.

Analysis of PAR-1 RNA Expression.

Immediately after sorting, RNA was extracted and purified using RNeasy (Qiagen, Chatsworth, Calif.) according to manufacturers' specifications. Genomic DNA contamination was removed through an on-column DNase digestion step. 20 ng of total RNA was amplified using ribo-SPIA based amplification (Nugen, Inc), labeled, fragmented and hybridized to HG-U133 Plus 2.0 GeneChips (Affymetrix). Microarray data was pre-processed using RMA (Irizarry et al., “Summaries of Affymetrix Genechip Probe Level Data,” Nucleic Acids Res 31:e15 (2003), which is hereby incorporated by reference in its entirety), and downstream analysis was performed using Bioconductor and R (Gentleman et al., “Bioconductor: Open Software Development for Computational Biology and Bioinformatics,” Genome Biol 5:R80 (2004), which is hereby incorporated by reference in its entirety). Informative probe sets were determined using FARMS, which uses probe level information as repeated measures to quantify the signal-to-noise ratio of each given probe set (Talloen et al., “I/Ni-Calls for the Exclusion of Non-Informative Genes: A Highly Effective Filtering Tool for Microarray Data,” Bioinformatics 23: 2897-902 (2007), which is hereby incorporated by reference in its entirety).

Differential gene expression analysis was performed using a linear model approach and employing an empirical Bayes method for calculation of statistical significance (Bioconductor, limma package) (Smyth, “Linear Models and Empirical Bayes Methods for Assessing Differential Expression in Microarray Experiments,” Stat Appl Genet Mol Biol 3:Article 3 (2004), which is hereby incorporated by reference in its entirety). The first model concentrated on identifying genes whose expression differed in the transformed glioma-derived A2B5⁺ tumor progenitor cells (TPCs) from the non-transformed epilepsy-derived A2B5⁺ glial progenitor cells. As such, samples were either designated tumor (A2B5⁺ cells from tumor, n=20), normal (A2B5⁺ cells from epilepsy, n=8), or tissue dissociates (n=8). Following fitting of this linear model, those probe sets were identified whose expression were significantly enriched or depleted in tumor progenitors by at least 3 fold change and statistically significant following 1% FDR adjustment of p-values. The second linear model focused on identifying those genes whose expression significantly varied within and between low and high-grade glioma A2B5⁺ TPCs. WHO grade II, oligodendroglioma, astrocytoma, oligoastrocytoma, and ganglioglioma were grouped as low grade tumors (LG, n=10) and WHO grade III and IV tumors (anaplastic astrocytoma, anaplastic oligodendroglioma, anaplastic oligoastrocytoma, GBM, small cell GBM, and gliosarcoma) were grouped as high grade (HG, n=10). Significantly varying genes were identified using the same criteria (3 fold change, 1% FDR) and each phenotype was compared back to their native A2B5 progenitor. In contrast, the third linear model focused on tumor phenotype specific differences. The linear model was designed with separate groups for each tumor subtype.

Real-Time Polymerase Chain Reaction Analysis.

Extracted total RNA was amplified using ribo-SPIA based whole transcriptome based amplification (NuGen). The expression of 95 cell type marker and pathway-specific genes was assessed using a 96-gene Taqman low-density array (TLDA) (Applied Biosystems). The relative abundance of transcript expression was calculated by ΔΔC analysis, and the expression data normalized to GAPDH. Genes whose expression was not detected in more than half of the RNA samples were excluded. Statistical analysis of TLDA gene expression data was then performed using Bioconductor, using a moderated t-test statistic with a 5% false discovery rate cut-off (Smyth, “Linear Models and Empirical Bayes Methods for Assessing Differential Expression in Microarray Experiments,” Stat Appl Genet Mol Biol 3:Article 3 (2004), which is hereby incorporated by reference in its entirety).

PAR-1 gene expression was also measured by PAR-1/F2R-directed RT-PCR. 100 ng total RNA was reverse-transcribed following manufacturer instructions (TaqMan Reverse Transcription Reagents, Applied Biosystems). 10 ng cDNA was used for each reaction. RT-PCR parameters were: 94° C. for 3 min, 94° C. for 45 min, 55° C. for 45 min, 72° C. for 45 min (40 cycles) followed by 1 cycle of 7 min at 72° C. F2R specific primers were 5′-GCCCGCAGGCCAGAATCAAA-3′ (SEQ ID NO: 2) and 5′-CGCCGGCTTCTTGACCTTCA-3′ (SEQ ID NO: 3) with an annealing temperature of 59.8° C. To verify that the same amounts of total mRNA were used for each RT-PCR, transcription of the housekeeping GAPDH gene was monitored. Amplification products were separated on 1% agarose gels stained with Syber safe (Invitrogen), and visualized under UV light. For each RT-PCR, a negative control PCR without the MultiScribe Reverse Transcriptase was included. For quantitative RT-PCR, individual primers and probes for F2R were obtained as Assays-on-Demand from Applied Biosystems. 10 ng of cDNA was used for each reaction. Statistical analysis was performed on log-transformed data and p-values calculated (1-way ANOVA followed by Tukey pairwise comparisons). P-values less than 0.05 were selected as significant.

Generation and Validation of PAR-1 Knock-Down Lentiviruses.

A set of lentiviral shRNA vectors containing 5 constructs with distinct target sequences was purchased from Open Biosystems (RHS4533-NM_{—}001992). PAR-1-induced silencing by transfecting glioma cell lines was first verified, with subsequent q-PCR for PAR-1 mRNA expression. Only selected and validated constructs were packaged for viral production. Subconfluent 293T cells were then co-transfected with an equimolar amount of PLKO-empty vector or PLKO-PAR-1 shRNA plasmids DNA, and a mixture of the packaging plasmids PAX2 (Addgene, Cambridge, Mass.) and vesicular stomatitis virus glycoprotein (VSVG), using Fugene HD (Roche Applied Science, Indianapolis, Ind.). Viral supernatants were collected at 48 and 72 hrs by ultra-centrifugation at 18,000 g for 3 hrs, aliquoted and stored at −80° C. The viral titer was determined by transduction of HT1800 cells with serial dilutions of the viral supernatant and colony counting after puromycin selection (1 μg/ml). To confirm gene knock-down and measure its efficacy, both PAR-1 mRNA and PAR-1 protein expression were assessed by quantitative-PCR and flow cytometry, respectively, six days after shRNA lentiviral transduction.

Cell Proliferation and Cell Cycle Analysis.

Six days after lentiviral-induced knock-down of PAR-1 in glioma cell lines or transduction with scrambled control lentiviruses, cells were incubated with EdU and subsequently stained using the Click-It™ Edu flow cytometry assay kit (Invitrogen, Grand island, NY, A10202). Briefly, cells transduced with either scrambled or PAR-1-KD lentiviruses were seeded in 6 well plates. Five days post-transduction, cells were collected, passaged using trypsin-LE, counted and replated in 6 well plates. 24 hours after plating, cells were treated with 10 μM of EdU for 30 to 60 minutes at 37° C., collected, and centrifuged at 400 RCF for 5 minutes for collection of nuclei. The pellets were resuspended in 0.5 ml of 0.03% Igepal CA-630 in 0.1% sodium citrate for 30 seconds, as described (Hamelik and Krishan, “Click-It Assay with Improved DNA Distribution Histograms,” Cytometry A 75:862-865 (2009), which is hereby incorporated by reference in its entirety). Cell suspensions were vortexed and 2.5 ml of Dulbecco's phosphate buffered saline (D-PBS) with 1% bovine serum albumin (BSA) was added. Nuclei were further stained with Click-It™ (Invitrogen, Grand Island, N.Y.) reaction mixture for 30 minutes. The cells were then spun, washed, and incubated with propidium iodide (PI) with RNase for 30 minutes at room temperature, according to the manufacturer's instructions. The experiments were repeated 3 times independently. Cells were then analyzed on a FACS ARIA flow cytometer (BD Biosciences) using FACS DIVA software and/or FlowJo. After forward scatter and side scatter gating to remove debris and clusters, PI was used to isolate whole nuclei and exclude DNA fragments. Values were reported as means±S.E.M. Statistical analysis was performed using 1-way ANOVA followed by Tukey's multiple comparison test with * p<0.05; ** p<0.01; ***p<0.001.

Orthotopic Transplantation.

To assess in vivo tumorigenicity, adult (5-10 week-old; 21-23 g) SCID/NOD and NSG mice (NOD/Shi-scid/IL-2Rγ^{null}) (Jackson Labs) were maintained in micro-isolator cages in a specific pathogen-free facility on a standard 12-hour night and day cycle. Injections were performed according to institutional guidelines. Dissociated glioma and TPC cell suspensions were diluted to a concentration of viable 20,000 to 100,000 cells/μl and placed on ice until transplantation. Animals were anesthetized with ketamine-xylazine and placed in a stereotaxic apparatus, and cells injected at 0.4 μl/min to the following coordinates: AP −0.98 mm from bregma; ML: +2.2 mm; DV:−2.2 mm. The syringe was left in place for 5 min following cell injection, and the wound then closed. After completion of the experiments, residual cells were grown in culture to validate their viability in vitro. Animals were subsequently examined for behavioral changes and weight loss, until the time of sacrifice, 4-6 weeks after injection. At that time, animals were terminally anesthetized, serially perfused via a transcardiac approach with saline solution and 4% paraformaldehyde, then their brains were removed, post-fixed for 2 h, serially cryoprotected in 6% and 30% sucrose, and frozen in cooled methyl-butane. The brains were serially sectioned at 14 m by cryostat; sections were stored frozen for subsequent immunohistochemical and histological analysis.

Histological and Immunohistochemical Analysis.

Sections were rehydrated in PBS and permeabilized for 15 min with PBS containing 0.1% saponin and 1% normal goat/donkey serum (NGS/NDS). Sections were washed three times in PBS and then incubated for 1 h with PBS containing 0.05% saponin and 10% NGS/NDS. The primary antibodies were diluted in PBS containing 0.01% saponin and 5% NGS/NDS and incubated overnight. After three washes with PBS, sections were further incubated with a solution of secondary antibodies (in PBS containing 0.01% saponin and 5% NGS/NDS) using AlexaFluor488 and 594-labeled secondary antibodies (1/500-1/1000, Molecular Probes). Sections were counter stained with 4′-6-diamidino-2-phenylindole (DAPI; Invitrogen), and examined using either an Olympus BX51 epifluorescence or Fluoview 100 confocal microscope. Adjacent sections were typically stained with hematoxylin and eosin for histological assessment. For immunohistochemical analysis of xenografts, transplanted cells were identified using antibody to human nuclei (HNA). In order to examine the neoplastic status of the cells, the expression of Ki67 and survivin (Andersen et al., “The Universal Character of the Tumor-Associated Antigen Survivin,” Clin Cancer Res 13:5991-5994 (2007), which is hereby incorporated by reference in its entirety) were assessed, with stereological estimation of incidence as previously described.

Thrombin and PAR-1 Specific Inhibitors.

Dabigatran etexilate (Pradaxa) was obtained as 150 mg capsules from Boehringer Ingelheim (Ingelheim, Germany) for in vivo experiments. Each capsule was freshly dissolved in 4 ml of acidic water to yield 37.5 mg/ml. Dabigatran etexilate was dosed depending of the body weight of the mouse, twice a day, using oral gavage. For in vitro analysis, the chemical compound dabigatran etexilate (BIBR-1048) was purchased from Cedarlane USA (S2154-5 mg) and dissolved in dimethylsulfoxide (DMSO) following manufacturer's instructions.

The selective PAR-1 antagonist SCH79797 was purchased from Tocris Bioscience (Ellisville, Mo., USA) and dissolved in dimethylsulfoxide (DMSO) following manufacturer's instructions. The selective oral PAR-1 inhibitor SCH 530348 (Vorapaxar) was purchased from Axon medchem (Axon 1755) and reconstituted following manufacturer's instructions.

Subcutaneous Heterotopic In Vivo Model.

Glioma cells and/or A2B5⁺ TPCs (1-4E⁺06 cells/animal) were collected, resuspended in 75 μl of media, and mixed (1:1) with Matrigel (9.8 mg/ml), prior to subcutaneous injection (150 μl, final volume) into the flanks of immunodeficient NOG mice (5-8 weeks) (1-4E⁺⁰⁶ cells/animal). Subcutaneous tumor growth was measured every other day morphometrically using vernier calipers, and tumor volumes were estimated according to the formula a²b/2, where a and b are the shorter and longer diameters of the tumor, respectively. Beginning 2 weeks post-transplantation, half the mice were dosed with dabigatran etexilate by gavage, twice a day, at 37.5 mg/kg. The remaining mice were gavaged at the same times with vehicle control (acidic water). Mice were sacrificed between 5 to 6 weeks after tumor cell injections, the tumor excised and fixed in a solution of paraformaldehyde 4% for further histological and immunohistochemical analysis.

### Example 1

### PAR-1 is Overexpressed in A2B5-Defined Glioma Tumor Precursor Cells (TPCs)

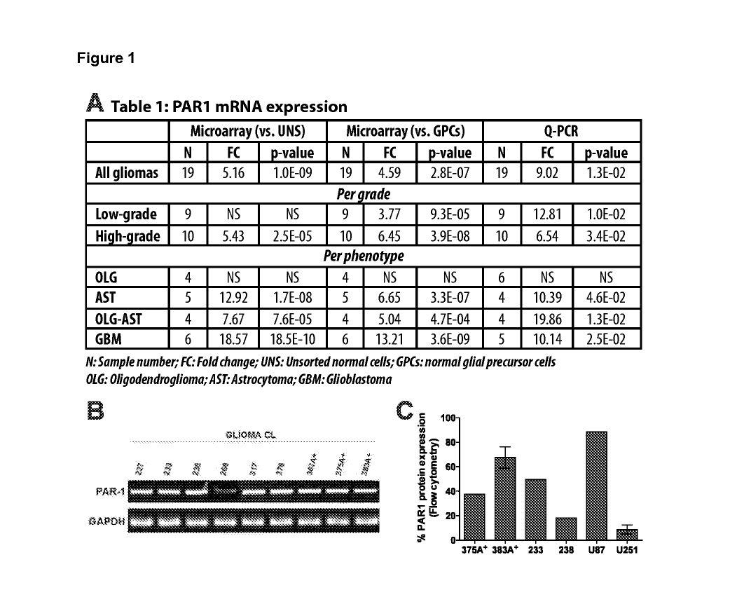

To identify new pathways to target for treatment of malignant glioma, the gene expression profile of glioma TPCs relative to their normal homologues using microarray analysis was assessed. This strategy was based on the expression of specific gangliosides recognized by the monoclonal antibody A2B5, which identifies normal glial precursor cells (GPCs) in the adult human brain (Nunes et al., “Identification and Isolation of Multipotential Neural Progenitor Cells from the Subcortical White Matter of the Adult Human Brain,” Nat Med 9:439-447 (2003), which is hereby incorporated by reference in its entirety). A2B5-defined glioma cells exhibited biological features of TPCs in vitro and in vivo. The expression profile ofA2B5-sorted glioma TPCs isolated from oligodendroglioma (n=4), oligoastrocytoma (n=4), astrocytoma (n=5), and glioblastoma multiforme (n=6) was further analyzed and compared directly to the expression profile of their normal adult A2B5⁺ GPCs homologues isolated from adult sub-cortical white matter and cortex (n=4, each), and/or unsorted normal adult cells (n=5) using microarray analysis (Affymetrix, U133 Plus 2.0 GeneChip). Those probe sets whose expression were significantly enriched or depleted in A2B5⁺ glioma TPCs by at least 3 fold change, using a moderated t-test statistic with 1% false discovery rate cut-off (q<0.05, linear modeling empirical Bayes test statistic) were identified. Using this method, a cohort of dysregulated genes was identified among which the thrombin receptor PAR-1 appeared as potently up-regulated by A2B5⁺ TPCs relative to their normal homologues, at every stage of glioma progression (FIG. 1A), including both low-grade and anaplastic astrocytomas, mixed oligo-astrocytoma and GBM (FIG. 1A).

To validate the array data, selected target genes were chosen for real-time Taqman q-PCR validation using a 96-gene Taqman low-density array (TLDA), following normalization to GAPDH and calculation of p-values from ACₜ values. Expression data was analyzed using a moderated t-test statistic with a 5% false discovery rate cut-off to compare glioma-derived A2B5⁺ tumor cells (n=19) to their non-tumor A2B5⁺ counterparts (n=5). Similar analysis was performed to compare differential gene expression associated with tumor grade and tumor phenotype. By doing so, an overexpression of PAR-1 in astrocytoma, mixed oligo-astrocytoma and GBM, in accordance with the microarray results (FIG. 1A) was confirmed. Using real-time PCR analysis, it was also found that PAR-1 was significantly overexpressed in the majority of glioma TPC lines, established from primary GBMs and cultured in serum-free media supplemented with FGF, EGF (20 ng/ml), and PDGF (10 ng/ml) (SFM) (FIG. 1B). To confirm that PAR-1 protein as well as mRNA was overexpressed, flow cytometry was performed and it was found that PAR-1 was overexpressed in the glioma GBM-derived TPC lines established from both A2B5⁺ sorted cells (375 and 383) and unsorted GBM cells (233, 238), as well as from widely-used established glioma lines (U87, U251) maintained in serum-containing media (FIG. 1C).

### Example 2

### PAR-1 Silencing Inhibits the Growth and Proliferation of Glioma Cells and TPCs

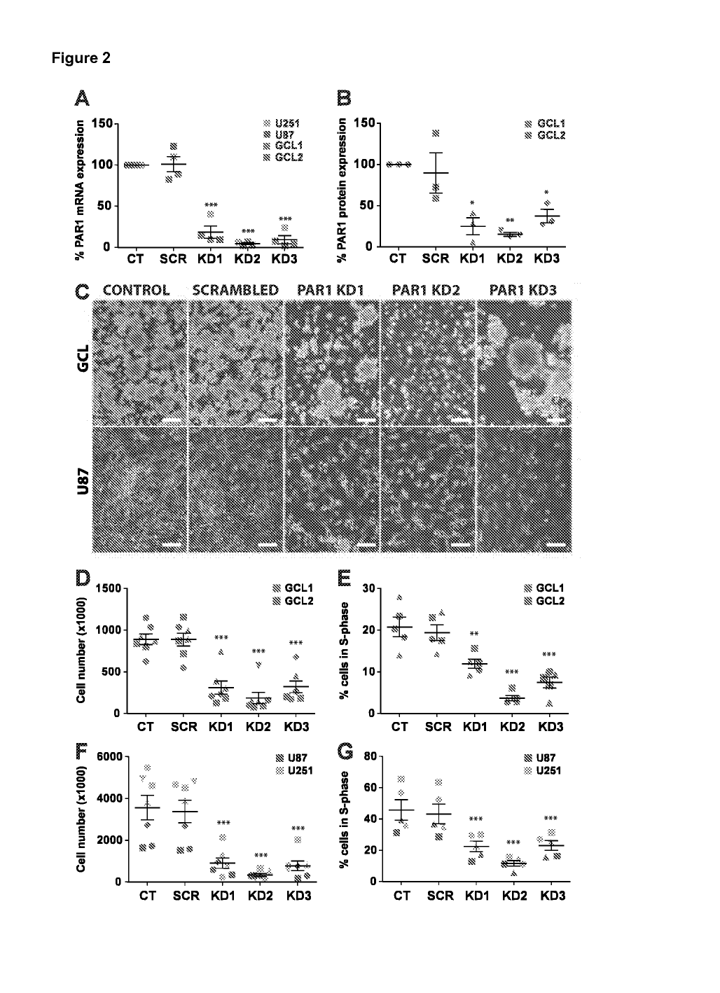

The robust overexpression of PAR-1 in A2B5-defined TPCs and glioma TPC lines prompted examination of its contribution to gliomagenesis. To determine whether glioma TPCs were dependent on PAR-1 signaling, the effect of lentiviral-induced knock-down of PAR-1 expression on glioma TPC lines growth in vitro was tested. It was found that PAR-1 gene and protein expression were both down-regulated in response by PAR-1-directed lentiviral shRNAi. Quantitative RT-PCR (FIG. 2A) and flow cytometry (FIG. 2B) were used to respectively assess mRNA and protein expression in both A2B5⁺ derived glioma TPCs, and in U87 and U251 glioma cell lines, 6 days after transduction with 3 different PAR-1 knock-down (KD) lentiviruses; all were compared to matched controls that included cells transduced with a scrambled lentivirus (SCR), as well as otherwise unmanipulated cells (CNTRL) (FIG. 2).

All 3 lentiviruses were effective in reducing the expression of both PAR-1 mRNA (FIG. 2A) and protein (FIG. 2B), relative to control lentivirus, in glioma TPC lines derived from both A2B5⁺ GBM cells and glioma cell lines (FIG. 2A). To determine if PAR-1 suppression influenced glioma genesis or progression, the effect of PAR-1 silencing on the growth of A2B5⁺ derived TPCs was then examined. It was found that lentiviral shRNAi knock-down of PAR-1 (PAR1 KD) significantly reduced the number of GBM-derived A2B5 TPCs in vitro, relative to both scrambled (SCR) shRNAi-transduced and non-transduced control (CT) cells, 6 days post-transduction (FIGS. 2C-2D, FIG. 2F).

On that basis, the effects of PAR-1-induced KD on cell proliferation and cell cycle progression was next investigated by analyzing EdU incorporation in association with propidium iodide (PI) staining. It was found that both A2B5⁺ TPCs and glioma lines subjected to PAR-1 KD by lentiviral shRNAi manifested fewer cells in S phase relative to SCR and CT cells (FIGS. 2E and 2G).

### Example 3

### PAR-1 Silencing Suppressed the In Vivo Expansion of Both Glioma TPCs and U87 Glioma Cells

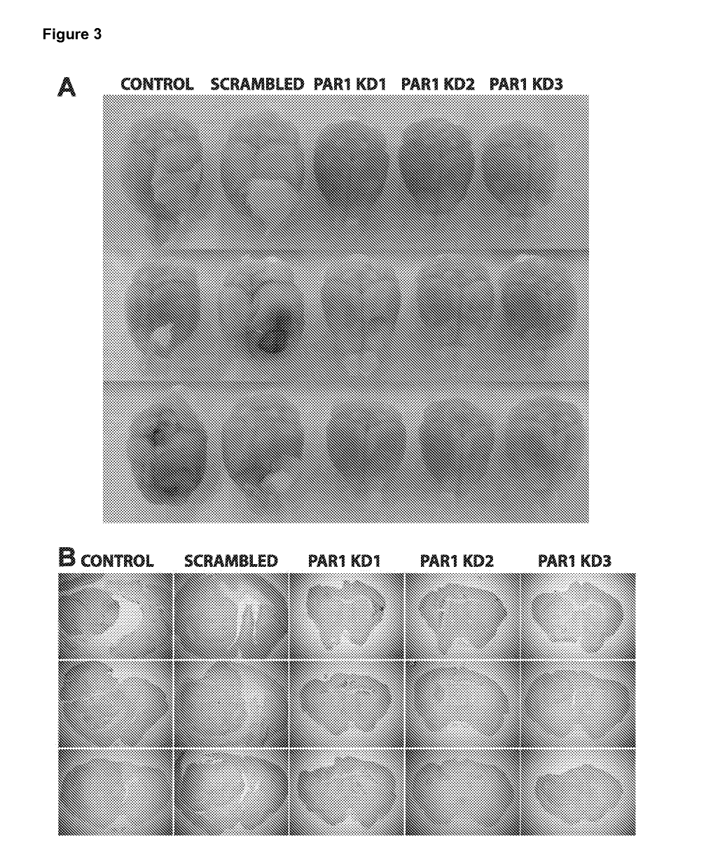

The potential anti-tumor effect of PAR-1 inhibition on the tumorigenic competence of glioma cells and A2B5⁺ TPCs in vivo was next evaluated. Given that U87 cells demonstrated the highest degree of PAR-1 expression (88.6±0.05%), PAR-1-shRNA expressing U87 cells, as well as untransduced control cells (CT) and U87 cells transduced with scrambled control lentivirus (SCR) were implanted intracranially into the brain of immunodeficient NOG mice, 6 days after lentiviral transduction (8×10⁴ cells/animal, n=3 each for PAR1 KD, SCR and CT cells). PAR-1 silencing dramatically suppressed the tumorigenicity of U87 cells, compared to both CT and SCR infected cells, as illustrated both on a macroscopic (FIG. 3A) and microscopic level (FIG. 3B) 4 weeks after transplantation. The observed anti-tumor effect of PAR-1 blockade was more prominent for both PAR1 KD1 and KD2 lentiviruses. For this reason, these constructs have been selected for subsequent experiments (FIG. 3B).

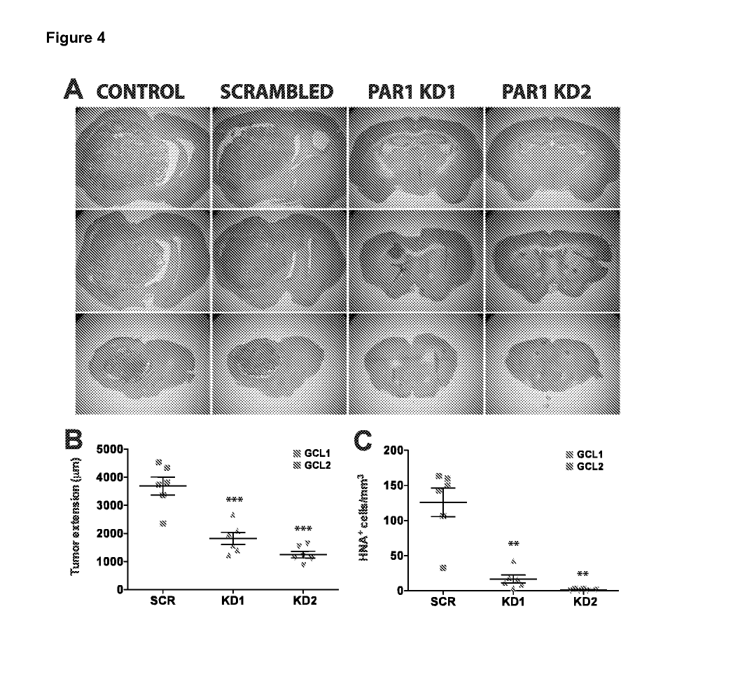

The effect of PAR-1 inhibition on the tumorigenicity of TPCs established from A2B5⁺ sorted cells derived from two WHO IV glioblastoma (2.8×10⁴ cells/animal, n=3 each for PAR1 KD, SCR and CT cells, for each of the 2 lines) was then examined. The cells were transplanted into the brain of immunodeficient mice, 6 days after lentiviral transduction. PAR-1 silencing inhibited the tumorigenicity of A2B5⁺ glioma TPCs, as demonstrated by a significant decrease in tumor bulk and extension along the antero-posterior axis for animals transplanted with shRNA-PAR1 KD TPCs, relative to control and SCR infected cells, when assessed at 6 weeks post-transplant (FIGS. 4A-4B). In addition, the number of human transplanted HNA⁺ cells, normalized to the total brain volume, was dramatically and significantly decreased in animals transduced with both PAR1 KD1 and KD2 lentiviruses relative to SCR shRNAi controls (FIG. 4C). These observations indicated that PAR-1 knockdown potently inhibited the growth, proliferation, and survival of glioma TPCs in vitro, as well as their tumorigenicity in vivo. Together, these data strongly suggest that PAR-1 plays a critical role in the initiation as well as the proliferative expansion of malignant glioma.

### Example 4

### Pharmacological Inhibition of PAR-1 Inhibits the Expansion of Glioma Cells and TPCs In Vitro

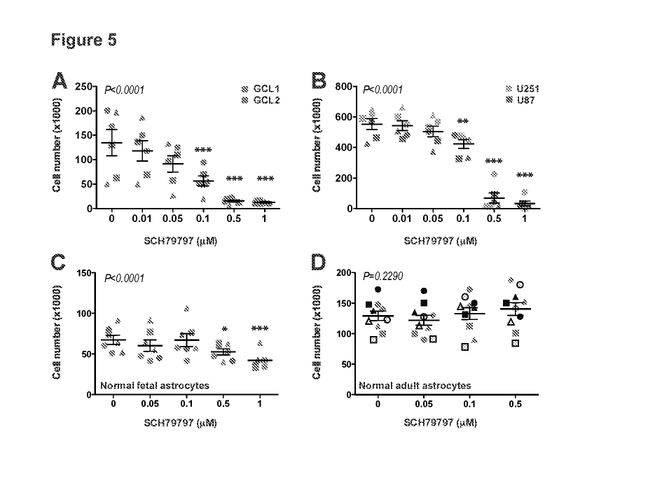

The various physiological and pathophysiological effects exerted by PAR-1 activation have led to the development of multiple selective PAR-1 antagonists over the years (Garcia-Lopez et al., “Thrombin-Activated Receptors: Promising Targets for Cancer Therapy?” Curr Med Chem 17:109-128 (2010), which is hereby incorporated by reference in its entirety). Among them, the pyrroloquinazoline SCH-79797 has been described as a potent anti-angiogenic in vivo (Tsopanoglou and Maragoudakis, “Inhibition of Angiogenesis by Small-Molecule Antagonists of Protease-Activated Receptor-1,” Semin Thromb Hemost 33:680-687 (2007); Zania et al., “Blockade of Angiogenesis by Small Molecule Antagonists to Protease-Activated Receptor-1: Association with Endothelial Cell Growth Suppression and Induction of Apoptosis,” J Pharmacol Exp Th 318:246-254 (2006), which are hereby incorporated by reference in their entirety). SCH-79797 also showed anti-proliferative effects, blocked cell migration and growth of several human and mouse cell lines (Kaufmann et al., “Thrombin-Mediated Hepatocellular Carcinoma Cell Migration: Cooperative Action Via Proteinase-Activated Receptors 1 and 4,” J Cell Physiol 211:699-707 (2007); Ma et al., “Proteinase-Activated Receptors 1 and 4 Counter-Regulate Endostatin and Vegf Release from Human Platelets,” Proc Natl Acad Sci USA 102:216-220 (2005), which are hereby incorporated by reference in their entirety). On that basis, it was further asked whether pharmacological inhibition of PAR-1 can slow the growth of glioma cells and A2B5⁺ TPCs. Adherent glioma cells grown in 10% Serum (U87, U251) and A2B5⁺ TPCs derived from primary GBM cell lines cultured in SFM were subjected to escalating doses of the PAR-1 antagonists SCH-79797. It was found that in all cell lines, SCH-79797 induced a significant concentration dependent inhibition of cell growth, compared to control cultures treated with vehicle alone (DMSO) (FIGS. 5A-5B). Given that PAR-1 has been shown to be expressed throughout the adult human brain, intensely in astrocytes and moderately in neurons (Junge et al., “Protease-Activated Receptor-1 in Human Brain: Localization and Functional Expression in Astrocytes,” Exp Neurol 188:94-103 (2004), which is hereby incorporated by reference in its entirety), the effects of SCH-79797 on normal astrocytes lines established from fetal and adult human brain was further examined. SCH-79797 was able to slow the growth of normal fetal astrocytes, however, a significant decrease in the number of fetal astrocytes was observed for SCH-79797 concentrations ≧0.5 μM, while a significant effect was observed on glioma cells and A2B5⁺ TPCs, starting at 0.1 μM (FIG. 5C). In addition, no significant differences were observed when SCH-79797 was administered to normal adult astrocytes (FIG. 5D).

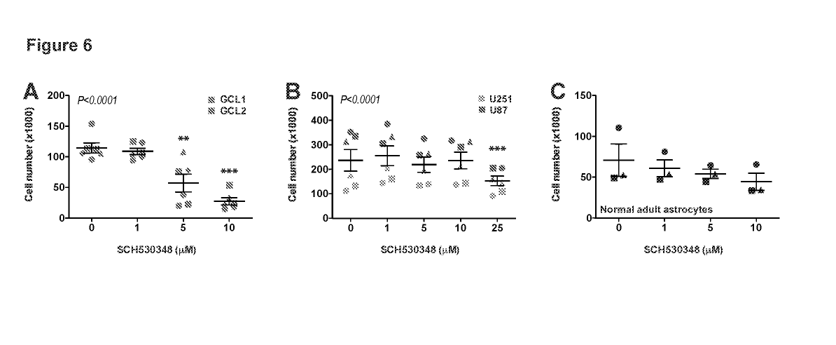

Given that the PAR-1 selectivity of SCH-79797 has been recently questioned (Di Serio et al., “Protease-Activated Receptor 1-Selective Antagonist Sch79797 Inhibits Cell Proliferation and Induces Apoptosis by a Protease-Activated Receptor 1-Independent Mechanism,” Basic Clin Pharmacol Toxicol 101:63-69 (2007), which is hereby incorporated by reference in its entirety), it was decided to duplicate the in vitro experiments using the non-peptide competitive PAR-1 antagonist SCH-530348 (Vorapaxar) which demonstrates excellent oral bioavailability and safety (Lee and Hamilton, “Physiology, Pharmacology, and Therapeutic Potential of Protease-Activated Receptors in Vascular Disease,” Pharmacol Ther 134:246-259 (2012), which is hereby incorporated by reference in its entirety). Indeed, SCH-530348 represents currently the most advanced and orally available specific PAR-1 antagonist, and has been evaluated in two large phase III clinical trials in patients with cardiovascular disease. Vorapaxar significantly reduced major cardiovascular thrombotic events in stable patients with a history of myocardial infarction suggesting that PAR-1 is a novel therapeutic target for long-term secondary prevention after myocardial infarction (Lee and Hamilton, “Physiology, Pharmacology, and Therapeutic Potential of Protease-Activated Receptors in Vascular Disease,” Pharmacol Ther 134:246-259 (2012); Siller-Matula et al., “Pharmacokinetic, Pharmacodynamic and Clinical Profile of Novel Antiplatelet Drugs Targeting Vascular Diseases,” Br J Pharmacol 159:502-517 (2010), which are hereby incorporated by reference in their entirety). To examine its effect on glioma cells and TPCs, SCH-530348 was administrated with escalating doses to glioma cells following the same paradigm previously described. It was found that SCH-530348 significantly reduced the growth of A2B5 TPCs derived from GBM, at concentrations ≧5 μM (FIG. 6A). In addition, a significant decrease in the number of glioma cells cultured in serum-supplemented media was noted at 25 μM (FIG. 6B). Importantly, normal adult astrocytes were not affected by SCH-530348 administration (FIG. 6C).

### Example 5

### Thrombin Inhibition Via Dabigatran Inhibits the Growth of Glioma TPCs In Vitro

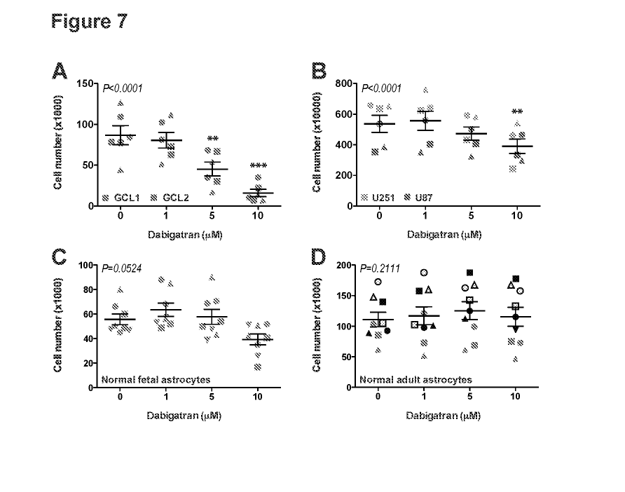

Given the availability of already approved and relatively safe drugs by which the thrombin may be antagonized, the effect of pharmacological inhibition of thrombin using the oral inhibitor dabigatran etexilate (dabigatran) in vitro was next examined. Dabigatran (marketed as Pradaxa) is an oral inhibitor of thrombin, approved for use in patients with or at risk of thrombotic disorders (Lee and Ansell, “Direct Thrombin Inhibitors,” Br J Clin Pharmacol 72:581-592 (2011); Stangier, “Clinical Pharmacokinetics and Pharmacodynamics of the Oral Direct Thrombin Inhibitor Dabigatran Etexilate,” Clin Pharmacokinet 47:285-295 (2008), which are hereby incorporated by reference in their entirety). Dabigatran has been reported to reduce breast cancer progression and inhibits the invasiveness of breast carcinoma cells in mice (DeFeo et al., “Use of Dabigatran Etexilate to Reduce Breast Cancer Progression,” Cancer Biol Ther 10:1001-1008 (2010), which is hereby incorporated by reference in its entirety). To examine whether dabigatran treatment might suppress the growth of glial neoplasms, glioma cells and TPCs were treated with escalating doses of dabigatran and counted 4 days after drug administration. Cultures of both glioma cells and A2B5⁺ TPCs treated with dabigatran exhibited decreased in vitro expansion in a dose dependent manner, such that a significant decrease in the number ofA2B5 TPCs was noted at concentrations ≧5 μM (FIG. 7A); this was notably lower than that at which the growth of adherent glioma cells was inhibited (≧10 μM) (FIG. 7B). In contrast, the number of both fetal and adult normal astrocytes cells was not significantly affected by increasing concentrations of dabigatran (FIGS. 7C-7D).

### Example 6

### Dabigatran Inhibits the In Vivo Expansion of Xenografted A2B5+Glioma TPCs

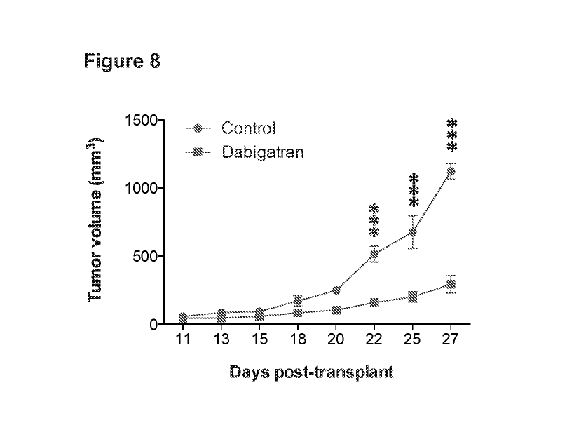

These observations indicated that dabigatran-induced thrombin inhibition decreased the in vitro expansion of glioma cells and A2B5⁺ TPCs. To translate these findings into a clinically relevant approach, the anti-tumor activity of dabigatran was further evaluated in vivo. Given that the blood-brain barrier (BBB) can constitute a substantial pharmacokinetic obstacle for a large majority of drugs, the effects of oral administration of dabigatran in mice bearing subcutaneous human glioma xenografts was assessed. A2B5⁺ TPCs derived from established GBM cell lines cultured in SFM were injected into the flank of immunodeficient NOG mice (2.5^{E+06} cells/animals, n=6 animals). Tumor volume was then measured every other day using vernier calipers. In humans, dabigatran is taken orally, twice daily, at a dose of 150 mg. Using appropriate methods of dose translation between species (Reagan-Shaw et al., “Dose Translation from Animal to Human Studies Revisited,” FASEB J 22:659-661 (2008), which is hereby incorporated by reference in its entirety), half of the mice were fed 37.5 mg/kg dabigatran twice daily via oral gavage (n=3), while half were given oral gavage of a vehicle control (acidic water; n=3), beginning 11 days after subcutaneous injection. Mice receiving dabigatran twice daily demonstrated a significant reduction in tumor volumes compared to vehicle controls (FIG. 8). The anti-tumor effect of dabigatran administration was significant 22, 24, and 26 days post-transplantation, corresponding to 11, 13, and 15 days of daily gavage.

### Discussion of Examples 1-6

In a comparison of gene expression by A2B5-defined glial tumor progenitor cells (TPCs) to glial progenitor cells derived from normal adult human brain, it was found that the F2R gene encoding PAR-1 was differentially up-regulated by TPCs isolated from primary gliomas at every stage of glioma progression, as well as by TPCs derived from established GBM lines. On that basis, and in light of the strong association of PAR-1 overexpression with other cancers, in this study it was asked whether PAR-1 was causally associated with glioma progression. It was first noted that lentiviral shRNAi knock-down of PAR-1 inhibited the expansion and proliferation of glioma TPCs both in vitro and in vivo after orthotopic transplantation. On that basis, it was then asked whether extant small molecule inhibitors of both thrombin and PAR-1, developed as anticoagulants, might suppress the progression or growth of malignant gliomas. It has been found by applicants that dabigatran, as well as the selective PAR-1 antagonists SCH79797 and SCH530348, indeed acted as potent inhibitors of glioma growth in vitro. Therefore, the antineoplastic activity of dabigatran in vivo, in mice given subcutaneous xenografts of A2B5-defined human glioma TPCs was next assessed. In this model as well, dabigatran significantly suppressed glioma growth, substantially reducing tumor bulk and burden relative to xenografted control mice treated with only vehicle. Together, these data identify the thrombin/PAR-1 axis as a substantial contributor to glioma progenitor expansion and hence to disease progression; as such, the abrogation of thrombin-dependent PAR-1 signaling may prove a promising strategy for the treatment of malignant glioma.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating glioma in a subject comprising:
selecting a subject having a glioma;
providing an inhibitor of binding between a PAR-1 receptor and a ligand of the PAR-1 receptor, and
administering the inhibitor to the selected subject under conditions effective to treat the glioma and/or prevent spread of tumor cells.

2. The method according to claim 1, wherein the inhibitor is a peptide, a peptide mimetic, a small molecule compound, or an antibody or binding portion thereof.

3-5. (canceled)

6. The method according to claim 1, wherein said glioma is located in the brain of the subject.

7. The method according to claim 1, wherein said glioma is located in the spinal cord of the subject.

8. The method according to claim 1, wherein said glioma is a Grade I glioma, a Grade II glioma, a Grade III glioma, or a Grade IV glioma.

9-11. (canceled)

12. The method according to claim 1, wherein the subject is a mammal.

13. The method according to claim 12, wherein the subject is a human.

14. The method according to claim 1, wherein said administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

15. The method according to claim 1, wherein the inhibitor is present in a pharmaceutical composition comprising the inhibitor and a pharmaceutically acceptable carrier.

16. The method according to claim 1, wherein the inhibitor is an inhibitor of direct physical binding between a PAR-1 receptor and a ligand of the PAR-1 receptor.

17. The method according to claim 1, wherein the inhibitor is an inhibitor of proteolysis of the PAR-1 receptor by a ligand.

18. The method according to claim 1, wherein the inhibitor is an inhibitor of PAR-1 receptor targeted protease.

19. A method of inhibiting proliferation of glioma cells and/or precursors thereof, said method comprising:
providing an inhibitor of binding between a PAR-1 receptor and a ligand of the PAR-1 receptor and
contacting the inhibitor with glioma cells and/or precursors thereof under conditions effective to inhibit proliferation of the glioma cells and/or precursors thereof.

20. The method according to claim 19, wherein said glioma cells are from a Grade I glioma, a Grade II glioma, a Grade III glioma, or a Grade IV glioma.

21-23. (canceled)

24. The method according to claim 19, wherein said method inhibits proliferation of glioma stem cells, glioma progenitor cells, or glioma cells.

25-26. (canceled)

27. The method according to claim 19, wherein the inhibitor is a peptide, a peptide mimetic, a small molecule compound, or an antibody or binding portion thereof.

28-30. (canceled)

31. The method according to claim 19, wherein the inhibitor is an inhibitor of direct physical binding between a PAR-1 receptor and a ligand of the PAR-1 receptor.

32. The method according to claim 19, wherein the inhibitor is an inhibitor of proteolysis of the PAR-1 receptor by a ligand.

33. The method according to claim 19, wherein the inhibitor is an inhibitor of PAR-1 receptor targeted protease.

34. (canceled)
